(19) United States
(12) Plant Patent Application Publication
(10) Pub. No.: US 2004/0133956 P1
(43) Pub. Date: Jul. 8, 2004

(54) Magnolia plant named 'JURmag1'

(76) Inventor: Mark C. Jury, North Taranaki (NZ)

Correspondence Address: Michael S. Doll, Fulwider Patton Lee & Utecht, LLP,, 200 Oceangate,, Suite 1550,, Long Beach, CA, 90802, US

(21) Appl. No.: 10/335,952

(22) Filed: Jan. 3, 2003

(51) Int. Cl. A01H 5/00

(52) U.S. Cl. PLT/223

(57) ABSTRACT

A new and distinct cultivar of Magnolia named ‘Black Tulip’ characterized by flowering as a very young plant; flowering for long period; dark plum flower color; and the flower held upright on the tree.

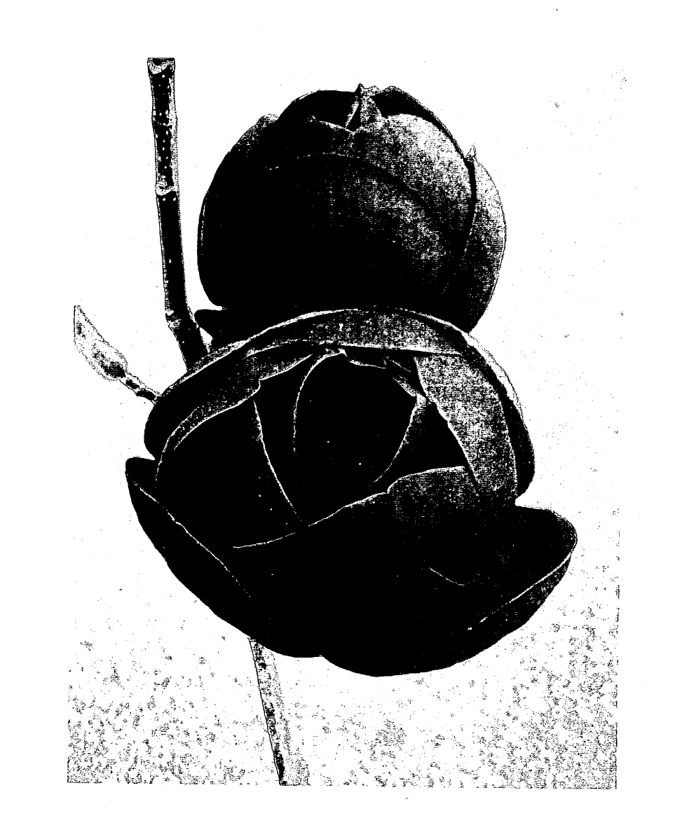

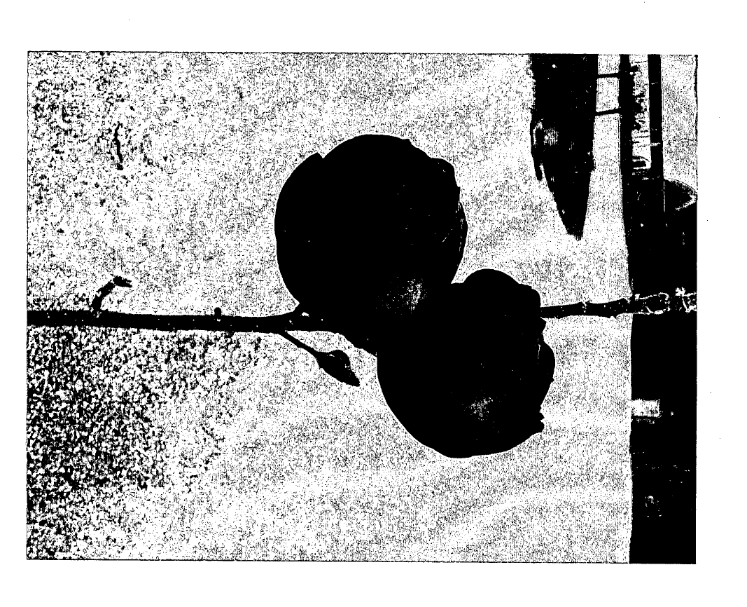

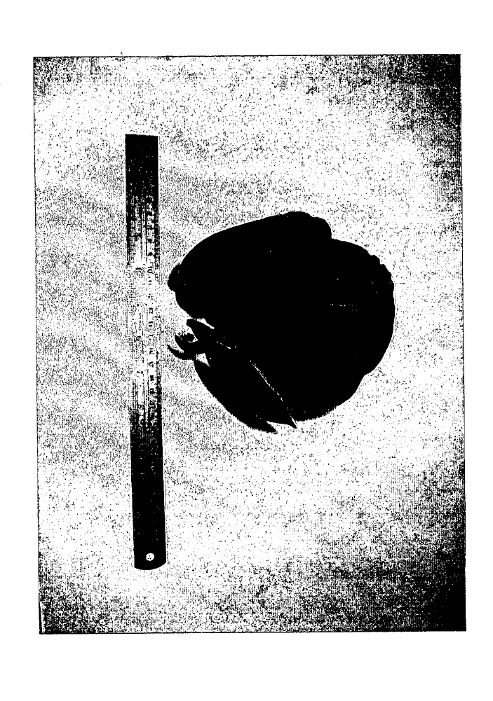

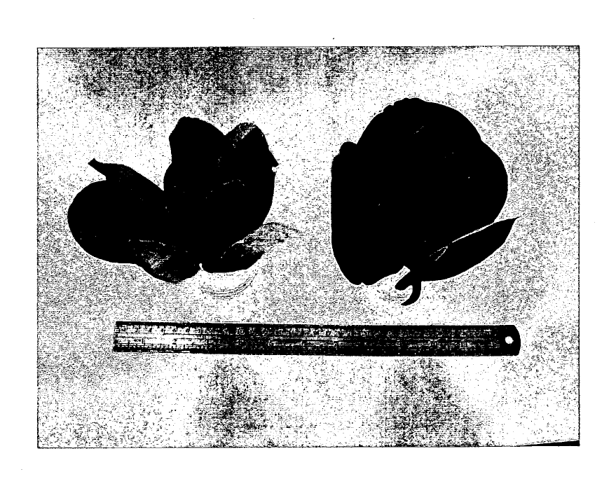

Latin Name of the Genus and Species of the Plant Claimed:

Magnolia soulangeana

Variety Denomination:

Black Tulip

### BACKGROUND OF THE INVENTION

The present invention relates to a new and distinct cultivar of Magnolia soulangeana referred to by the variety denomination ‘Black Tulip’.

The new cultivar was isolated by the inventor, Mark C. Jury, at North Taranaki in New Zealand, by seedling selection following controlled pollination of the maternal parent Magnolia ‘Vulcan’ (unpatented) with the paternal parent Magnolia ‘lolanthe’ (unpatented). The cross was made in 1986. The new cultivar was selected from the progeny of the stated cross by the inventor with the selection being primarily on the basis of flower color and form.

Following selection of the seedling, the cultivar was propagated from vegetative cuttings. Following several generations of propagated cuttings, uniformity and stability of the selected characteristics was observed. Horticultural examination of the present cultivar has demonstrated that the 20 combination of characteristics herein disclosed for Magnolia ‘Black Tulip’ are firmly fixed and are retained through successive generations of asexual reproduction.

The following table of characteristics indicates the manner in which the new cultivar is distinct from both Magnolia ‘lolanthe’ and Magnolia ‘Vulcan’. The comparison cultivar, Magnolia ‘Liliflora Nigra’, and Magnolia ‘Vulcan’ are the closest cultivars known to the applicant, but are quite different as shown in Table 1.

TABLE 1

| | | | | Comparison |
|---|---|---|---|---|
| | | | | Cultivar |
| | | | | Magnolia |
| | | Magnolia | Magnolia | hybrid |
| | Magnolia | Hybrid | Hybrid | ‘Liliflora |
| | ‘Black Tulip’ | ‘lolanthe’ | ‘Vulcan’ | Nigra’ |
| Flower Color | Deep | Mauve Pink | Wine Red | Dark |
| | Burgundy | | | Maroon |
| Flower Size | to 15 cm | to 25 cm | to 20 cm | |
| Plant Height | to 8 m | to 6 m | to 6 m | 4 to 6 m |
| Plant Shape | Pyrimadical | Rounded | Upright | Rounded |
| Plant Width | to 4.5 m | to 6 m | to 6 m | to 4 m |
| Trunk | Single trunk | Single trunk | Single trunk | Multi-trunk |

The following table of characteristics further indicates the manner in which the new cultivar is distinct from Magnolia ‘Vulcan’.

TABLE 2

| DESCRIPTION | Magnolia ‘Vulcan’ | Magnolia ‘Black Tulip’ |
|---|---|---|
| Petal Length | 70 mm | 110 mm |
| Petal Width | 60 mm | 95 mm |
| Petal shape | Elliptic | Obovate and Concave |
| Petal Margin | Entire | Entire |
| Petal RHS inside | RHS Red-Purple 57D | RHS Red-Purple 64A |
| Petal RHS outside | RHS Red-Purple 60C | RHS Red-Purple 61A |
| Petal Numbers | 12 | 12 |
| Flower shape | Saccate/solitary | Urceolate/solitary |
| Flower size | Approximately | Approximately |
| | 80 mm × 170 mm | 100 mm × 120 mm |
| Configuration | Loose whorl of 3 | Tight whorl of 3 |
| Fragrance | None detected | Medium fragrance |
| Style RHS | RHS Red-Purple 71A | RHS Red-Purple 63B |
| Stamen Numbers | 122 | 146 |
| Anther Color RHS | RHS Red-Purple 71A | RHS Red-Purple 71A |
| Filament Color RHS | RHS Yellow 11B | RHS Yellow-Orange 22B |
| Stamen Length | 13 mm | 16 mm |
| Stamen width | 1 mm | 1.8 mm |
| Pedicel | Short pubescent | Short pubescent |
| Bud sheath | Spathiate pubescent | Spathiate pubescent |
| Bud RHS color | Yellow-Green 146D with | Yellow-Green 146D |
| | splash of Red-Purple | |
| | 64B | |

### BRIEF DESCRIPTION OF THE INVENTION

The following traits have been repeatedly observed and are determined to be basic characteristics of Magnolia ‘Black Tulip’, which in combination distinguish this Magnolia hybrid as a new and distinct cultivar:

1. flowering as a very young plant;

2. flowering for long period;

3. dark plum flower color; and

4. flower held upright on the tree.

The following observations, measurements, and values describe the new cultivar as grown in Silvan, Australia under conditions which approximate those generally used in commercial practice.

### BRIEF DESCRIPTION OF THE DRAWINGS

The accompanying color photographs show the unique characteristics of the new cultivar of Magnolia ‘Black Tulip’, with colors as accurate as is possible with color photographs. The plants depicted in these photographic drawings are 3 years old. The first photographic drawing is a top view of a flowering ‘Black Tulip’ stem. The second photographic drawing is a side view of a flowering ‘Black Tulip’ stem with a bud. The third photographic drawing shows an individual ‘Black Tulip’ flower. The fourth photographic drawing shows a side by side comparison of a ‘Vulcan’ flower and a ‘Black Tulip’ flower.

### DETAILED BOTANICAL DESCRIPTION

Color references are made to The Royal Horticultural Society Colour Chart (RHS) except where general colors of ordinary significance are used. Colors are approximate as color depends on horticultural practices such as light level and fertilization rate among other things.

### ORIGIN

Seedling from breeding program.

### PARENTAGE

Maternal parent: Magnolia hybrid ‘Vulcan’.

Paternal parent: Magnolia hybrid ‘lolanthe’.

### PROPAGATION

Plants have been selected from the original seedling and propagated vegetatively by grafting in New Zealand.

### PLANT

Growth rate: Average.

Length of flowering season: Long.

Time of flowering season: Medium.

Density: Medium.

Growth type: Upright.

Height: 8 m.

Width: 5 m.

Stem diameter: 200 mm.

### LEAF

Color: Upper — RHS Green Group — 146A Under — RHS Green Group — 146B.

Length: 150 mm.

Width: 100 mm.

Apex: Acuminate.

Margin Entire.

Mid rib: RHS Yellow Group — 11B.

Texture: Glaucous on top, pubescent underneath.

Shape: Ovate.

Petiole: 20 mm in length; RHS Yellow/Green Group — 146B.

### STEM

Branching arrangement: Open.

Surface texture: Smooth.

Lenticels: Small; RHS Yellow Group — 11D.

### FLOWER

Number of flowers: Many.

Floret length: 100 mm.

Lastingness of bloom: Approximately 8 days.

Fragrance: Medium.

Flower shape: Ureolate/solitary.

Flower size: Approximately 100 mm×120 mm.

Configuration: Tight whorl of 3.

Flower profile upper: Flat.

Flower profile lower: Convex.

Color midzone outside: RHS Red Purple Group — 61A.

Midzone inside: RHS Red Purple Group — 61A.

Margin inside: RHS Red Purple Group — 64A.

Buds:

Bud shape.—Ovate.

Bud sheath.—Spathiate.

Length.—50 mm.

Width.—20 mm.

Color.—Dark grey pubescence present, RHS Yellow-Green Group — 146D.

Petals:

Number of petals.—12.

Petal size.—small.

Petal width.—95-100 mm.

Petal length.—110 mm.

Petal arrangement.—Overlapping.

Petal shape.—Obovate and concave.

Petal margin.—Entire.

Petal color inside.—RHS Red-Purple Group — 64A.

Petal Color outside.—RHS Red-Purple Group — 61A.

Pedicels:

Characteristic.—short pubescent.

Pedicel length.—15 mm.

Pedicel width.—10 mm.

Pedicel color.—RHS Yellow Group — 146D.

Calyx:

Sepal extension.—Weak.

Sepal length.—100 mm.

Stamen:

Stamen number.—146.

Length.—16 mm.

Width.—1.8 mm.

Filament color.—RHS Red Purple Group — 71A.

Anther color.—RHS Yellow Orange Group — 22B.

Basal spot: RHS Red Purple Group — 71A.

Seed vessel characteristics: Small, red in color.

Buds:

Bud shape.—Ovate.

Length.—50 mm.

Width.—20 mm.

Color.—Dark grey, RHS Yellow Green Group — 146D, pubescence present.

1. A new and distinct Magnolia plant named ‘Black Tulip’, as illustrated and described herein.
